(19) United States
(12) Patent Application Publication
(10) Pub. No.: US 2020/0360480 A1
(43) Pub. Date: Nov. 19, 2020

(54) METHOD OF TREATING IDIOPATHIC THROMBOCYTOPENIA PURPURA (ITP) WITH ROMIPLOSTIM

(71) Applicant: AMGEN INC., Thousand Oaks, CA (US)

(72) Inventors: Michael TARANTINO, Washington, IL (US); James BUSSEL, New York, NY (US); Melissa EISEN, Oak Park, CA (US); Nancy CARPENTER, Carterton, Oxfordshire (GB); Xuena WANG, Manlius, NY (US); Susanna MAC, Agoura Hills, CA (US)

(73) Assignee: AMGEN INC., Thousand Oaks, CA (US)

(21) Appl. No.: 16/771,641

(22) PCT Filed: Dec. 6, 2018
(86) PCT No.: PCT/US2018/064355

§ 371 Date: Jun. 10, 2020

Related U.S. Application Data
(60) Provisional application No. 62/596,020, filed on Dec. 7, 2017.

(51) Int. Cl. A61K 38/19; A61P 7/00

(52) U.S. Cl. CPC A61K 38/196; A61P 7/00

(57) ABSTRACT

The present invention concerns a method of treating idiopathic thrombocytopenia purpura (ITP) in a patient having ITP, which comprises: (a) administering romiplostim weekly to the patient; (b) increasing the weekly dose until a platelet count of at least about 50 to 200×10⁹/L is reached; (c) decreasing the weekly dose of romiplostim if the platelet count remains ≥200×10⁹/L for two consecutive weeks; (d) discontinuing romiplostim if the platelet count has remained ≥200×10⁹/L for two consecutive weeks when the weekly dose is 1 μg/kg or the platelet count is ≥400×10⁹/L; and (e) if a platelet count ≥200×10⁹/L is reached within the first 4 to 12 weeks of treatment, maintaining a treatment-free period of at least about 24 weeks during which the patient receives no romiplostim.

### BACKGROUND OF THE INVENTION

Primary ITP is an autoimmune disorder characterized by suboptimal platelet production and accelerated platelet destruction, mediated by both antibodies and T cells (Nugent et al, 2009). ITP in adults often has a chronic course, presenting with an increased risk of bruising and cutaneous bleeding, a reduced quality of life and, rarely, serious bleeding (Cines & McMillan, 2005).

The principal aim of treatment in these patients is durable improvement in platelet counts without on-going treatment. Current treatments include corticosteroids, anti-D immunoglobulin, and intravenous immunoglobulin (IVIg) first-line with romiplostim, eltrombopag, rituximab, and splenectomy second-line. These first-line treatments can produce platelet responses in a majority of patients, but a response may only be observed for weeks to months (Provan et al, 2010). For second-line treatments such as rituximab, 15-20% of patients may have complete response for up to 5 years. Splenectomy has a long-term response rate of approximately two-thirds (Provan et al, 2010).

The benefits of these treatments must be weighed against their potential risks, as adverse effects are associated with all first- and second-line treatments. Corticosteroids have well-recognized side effects, so clinicians often seek steroid-sparing approaches. IVIg and anti-D immunoglobulin are associated with infusion reactions, headaches, and hemolytic anemia. Immunosuppressive agents are associated with an increased risk of infection. These adverse effects as well as low rates of response limit the usefulness of these approaches.

Romiplostim is a thrombopoietin (TPO) receptor agonist that has been shown to increase and sustain platelet counts for up to 5 years and decrease the incidence of bleeding, splenectomy, treatment failure and rescue medication use (Kuter et al, 2008, 2010, 2013). Romiplostim is approved in various regions of the world for the treatment of chronic ITP in adults. It is approved for use in adults in the United States for treatment of chronic immune thrombocytopenia insufficiently responsive to corticosteroids, immunoglobulins, or splenectomy, and in Europe for those who have had a splenectomy and are refractory to other treatments or as second-line treatment in non-splenectomized patients for whom surgery is contraindicated. Romiplostim is administered at a starting dose of 1 μg/kg with weekly doses adjusted by 1 μg/kg to achieve and maintain a platelet count 50-200×10⁹/L.

Data is emerging on the use of romiplostim in children with chronic immune thrombocytopenia. Treatment of children with chronic immune thrombocytopenia is often guided by data from adult studies, pilot studies in children, and expert opinion. There have been three large randomized controlled studies in children with ITP that have demonstrated that treatment with TPO receptor agonists was associated with greater efficacy compared to placebo with no surprising or unexpected adverse events. However, data on long-term effects of treatments in children with ITP are lacking.

Romiplostim can induce platelet responses in approximately 80-90% of patients with ITP (Bussel et al, 2009; Kuter et al, 2010), but long-term treatment may be required to maintain platelet responses. Long-term treatment, however, may be associated with compliance issues and substantial costs (Ghanima et al, 2012).

Emerging data suggest that certain patients, many of whom had relapsed or refractory disease, may achieve lasting remission after discontinuation of romiplostim (Vlachaki et al, 2011; Ghadaki et al, 2013; Thachil et al, 2013; Mahevas et al, 2014; Provan et al, 2014; Tarantino et al. 2016, Tarantino et al, ASH 2017, abstract 14 as at: https://ash.confex.com/ash/2017/webprogram/Paper100126.html). Newland et al., 2016, conducted a systematic prospective evaluation of remission with romiplostim but ultimately concluded that no statistically significant predictors of remission were found when examining other demographic or baseline characteristics; however, higher mean platelet count during the first two months of treatment was associated with remission. The field would benefit from discovery of predictors of remission and methods of treatment exploiting such predictors.

### SUMMARY OF THE INVENTION

In the study underlying this specification, a platelet count ≥200×10⁹/L is reached within the first 4 to 12 weeks of treatment is found to be a predictor of remission, which is defined in the study as a treatment-free period of at least 24 weeks during which the patient receives no romiplostim or other ITP medication. Accordingly, this invention relates to a method of treating ITP in a patient having ITP, which comprises (a) administering romiplostim weekly to the patient; (b) increasing the weekly dose until a platelet count of at least about 50 to 200×10⁹/L is reached; (c) decreasing the weekly dose of romiplostim if the platelet count remains ≥200×10⁹/L for two consecutive weeks; (d) discontinuing romiplostim if the platelet count has remained ≥200×10⁹/L for two consecutive weeks when the weekly dose is 1 μg/kg or the platelet count is ≥400×10⁹/L; and (e) if a platelet count ≥200×10⁹/L is reached within the first 4 to 12 weeks of treatment, maintaining a treatment-free period of at least about 24 weeks during which the patient receives no romiplostim.

The invention also concerns a method as described above that comprises administering an initial dose of 1 μg/kg of romiplostim to the patient.

The invention further concerns a method as described above that comprises maintaining the weekly dose so long as the platelet count is within about 50 to 200×10⁹/L.

The invention further concerns a method as described above wherein the platelet count is ≥200×10⁹/L in the first four weeks of treatment.

The invention further concerns a method as described above wherein the increase in the dose of romiplostim is in increments of 1 μg/kg weekly.

The invention further concerns a method as described above wherein the dose in step c is decreased in increments of 1 μg/kg.

The invention further concerns a method as described above wherein the patient's platelet count remains ≥50×10⁹/L during the treatment-free period.

The invention further concerns a method as described above wherein the patient receives no ITP medications during the treatment-free period.

### BRIEF DESCRIPTION OF THE FIGURES

FIG. 1 shows a study flow chart. Of the 21 patients shown who entered the first extension of the study, one withdrew consent before treatment. Of the 66 patients who rolled into the second extension, one withdrew consent before treatment.

FIG. 2 shows the disposition of the patients who entered the study. Of the three patients who discontinued romiplostim per the protocol, two had remission and one had platelet counts <30×10⁹/L despite 10 weeks on 10 μg/kg. The 37 patients who completed romiplostim treatment received the drug until the study ended in January 2017, 12 months after the last patient enrolled.

FIG. 3A shows the romiplostim dose over time. The median (Q1, Q3) dose appears on the y-axis with the number of weeks in the study on the x-axis. FIG. 3B shows the platelet counts over time. The median (Q1, Q3) platelet count appears on the y-axis with the number of weeks in the study on the x-axis.

FIG. 4A shows bleeding over time for all patients and for those with grade ≥2. FIG. 4B shows grade ≥2 bleeding over time.

FIGS. 5A-5D show data on remission. FIG. 5A shows the probability of achieving remission plotted against the time taken to achieve remission, with the number of patients at risk at each time point also shown along the x-axis. FIG. 5B shows the number of patients achieving remission grouped by the duration of remission found in the study. FIGS. 5C and 5D show the dosage administered and platelet count achieved by two individual patients over the time of the phase 1/2, the first extension and the second extension studies.

FIG. 6 shows hazard ratios and p-values for selected remission characteristics.

FIGS. 7A-7M show remission data as described for FIGS. 5C and 5D for 13 additional patients.

### DETAILED DESCRIPTION OF THE INVENTION

TPO receptor agonists are not a perfect treatment option for ITP, as not every patient responds and those who do are not always able to maintain their responses. The subject invention is based on the largest study to date in children with TPO receptor agonists, with over 182 patient-years of exposure, or 2.8 years per patient, for 65 patients. Importantly, approximately 1/4 of patients were able to discontinue ITP treatments and still maintain hemostatic platelet counts. The data described in this manuscript are extensive, both in patient number (n=65) and treatment duration (up to 7 years), and show that romiplostim has an efficacy and safety profile that seems similar to that seen in adults.

The objectives of the study were to describe the safety and efficacy of long-term use of romiplostim in children with ITP, with the incidence of adverse events as the primary endpoint. Secondary endpoints included evaluation of long-term platelet responses, bleeding, reduced use of concurrent ITP medications (both continuation of medications from baseline as well as rescue medications), and a post hoc endpoint of maintaining platelet counts ≥50×10⁹/L for 6 months with no ITP medications, including romiplostim (here defined as remission).

### Methods

### Patients

Eligible patients had completed a previous romiplostim ITP study^{6,7} (Bussel et al., 2011; Tarantino et al., 2016) and were ≤18 years of age at time of enrollment. Exclusion criteria included a history of a bone marrow stem cell disorder, venous or arterial thrombotic or thromboembolic event, systemic lupus erythematosus, Evans syndrome, or other secondary causes of thrombocytopenia. The studies were conducted in compliance with all regulatory obligations and institutional review board and informed consent regulations at each investigational site. All patients or their legal representatives provided written informed consent and assent.

### Procedures

Patients were recruited from 28 sites in the United States, Canada, Spain, and Australia. The study (clinicaltrials.gov identifier NCT01071954) ran from 30 Dec. 2009 (first subject enrolled) to 12 Jan. 2017 (last patient last visit). During the study, patients received weekly subcutaneous romiplostim, starting with the same dose as the final dose in the parent study or 1 μg/kg (if previously on placebo or ≥24 weeks since the last dose). The dose was adjusted weekly in 1 μg/kg increments between 1-10 μg/kg to target platelet counts of 50-200×10⁹/L. Patients could continue to receive other ITP medications (e.g., corticosteroids, danazol, or azathioprine) that had been at a stable dose and schedule before study start. These additional medications could be reduced or discontinued per patient and physician preference after platelet counts exceeded 50×10⁹/L.

Patients could receive rescue medications when platelet counts were below 10×10⁹/L, when there was bleeding or wet purpura, or when deemed medically necessary by the investigator (e.g., prior to a trip or procedure). Rescue medications were defined as any medications administered to increase the platelet count and included intravenous immunoglobulin G (IVIg), anti-D, platelet transfusions, steroids, and antifibrinolytics (e.g., epsilon aminocaproic acid, tranexamic acid).

### Assessments, Outcomes, and Statistics

Platelet counts, concurrent medications, and adverse events were assessed at each visit. Samples were collected for complete blood counts and blood chemistry every 4 weeks, and physical examinations were performed at week 1 and every 12 weeks thereafter. Efficacy outcomes included platelet counts and platelet response (platelet count ≥50×10⁹/L without rescue medication use in the past 4 weeks). Platelet count data within 4 weeks of the use of rescue medications were excluded from the continuous summaries. Subjects who did not have weekly platelet counts due to self-administration or other reasons had their platelet counts carried forward as long as no rescue medication was used within 4 weeks of the missed visit. Other efficacy assessments included the proportion of patients using other ITP medications and the proportion of patients who required rescue medication. Remission was defined as platelet count ≥50×10⁹/L in the absence of all ITP medications including romiplostim for a period of at least 24 weeks.

### Safety

Safety assessments included review of adverse events (including bleeding), physical examination, vital signs, serum chemistries, complete blood count, platelet count, and antibody status. Samples were tested for binding antibodies against romiplostim and binding antibodies against TPO; any samples positive for binding antibodies against either were further tested for neutralizing antibodies. Medically significant adverse events considered to be treatment-related by the investigator were followed up until they resolved or were considered stable. For any adverse event, the investigator assigned the following attributes: description; dates of onset and resolution; severity; assessment of relatedness to treatment, other suspect drug, or device; and action taken (e.g., discontinuation of romiplostim, treatment with another medication, hospitalization, etc.). The severity of toxicities was assessed per the Standard Common Terminology Criteria for Adverse Events (CTCAE) version 3.0 grading of adverse events: 1 mild, 2 moderate, 3 severe, 4 life-threatening, 5 fatal. Serious adverse events included any adverse events that were fatal, life-threatening, required in-patient hospitalization, or prolonged existing hospitalization. Thromboembolic events were assessed as part of the overall adverse event evaluation. Bone marrow aspirates and biopsies were not required at any time, but could be performed at the investigator's discretion; for example, if there were abnormalities in the peripheral blood smear such as nucleated red blood cells or tear drop cells or if a loss of response to romiplostim occurred despite increasing doses. Blood samples for assays for antibodies to TPO and romiplostim were collected at week 1, week 52, annually, and at the end of the study.

### Data Analyses

Statistical analyses were descriptive. Categorical endpoints were summarized by the number and percentage of patients in each category. Continuous endpoints were summarized by number of patients, mean, standard deviation, median, and 25th percentile (Q1) and 75th percentile (Q3), with minimum and maximum values. Adverse events were also summarized as number of events and rate per 100 years of exposure.

Proportional hazards models were used to evaluate potential prediction factors for remission with subjects without remission censored at their final platelet count. For the univariate model, each potential factor was considered alone (analogous to a log-rank test). In the event that the assumption of proportional hazards was found to be violated, non-parametric tests (Fisher's exact test for categorical variables and Kruskal-Wallis test for continuous variables) were used instead. For the multivariate models, a forward stepwise selection criteria was used with significance levels for entry and exit set at 0.05.

### Results

### Demographics and Disposition

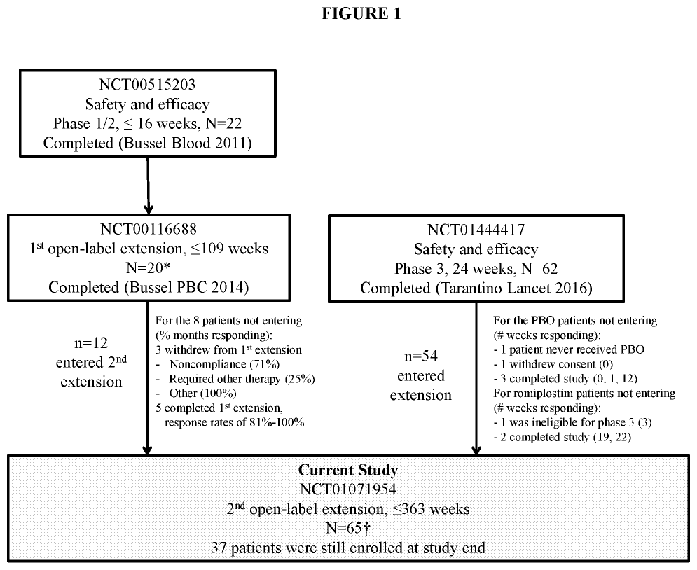

The flow of patients from the parent studies is shown in FIG. 1. Sixty-six patients entered this extension; 1 withdrew consent before treatment and 65 received romiplostim for ≤7 years. Fifteen of these patients had received placebo previously and this study was their first exposure to romiplostim. At baseline, median (min-max) age was 11 (3-18) years; 56% were female; 61% were white, 14% African American, 14% Hispanic/Latino, 9% Asian, and 3% other. Median (min-max) baseline platelet count was 27.5 (2-458)×10⁹/L (Table 1); patients could roll into this study without interruption of romiplostim dosing from their previous study. Past ITP treatments included IVIg, anti-D, corticosteroids, and rituximab; 9% had had prior splenectomy (Table 2).

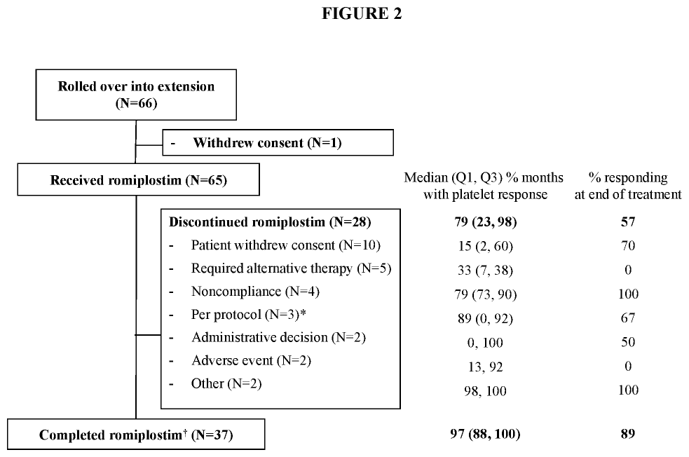

Reasons for discontinuing romiplostim (n=28, 42%) included consent withdrawn (n=10), required other therapy (n=6), noncompliance (n=4), per protocol (n=3), administrative decision (n=2), adverse event (n=2), and treatment not needed (n=1). The adverse events were asthenia, headache, dehydration, and vomiting in one patient and anxiety in the other; per the investigators, these adverse events were not considered treatment-related. Thirty-seven (56%) patients received romiplostim until study end (FIG. 2).

### Romiplostim Exposure

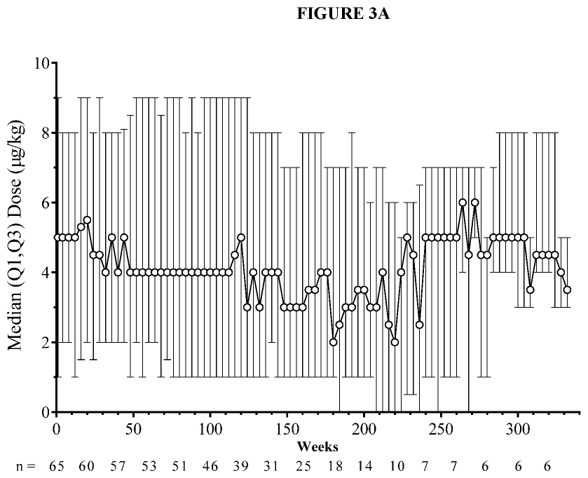

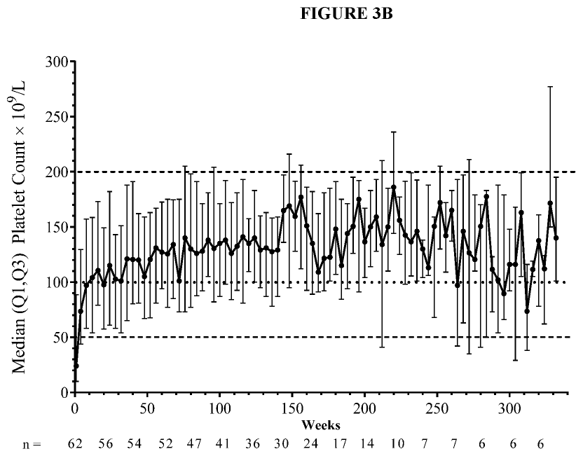

Median (min-max) romiplostim treatment was 135 (5-363) weeks, or a median of 2.6 years per patient, with a total of 182 patient-years. Median (min-max) average weekly]romiplostim dose was 4.8 (0.1-10) μg/kg, including escalation to a stable dose. The mean maximum weekly romiplostim dose was 6.9 μg/kg and the median maximum weekly dose was 8.0 μg/kg. Twenty patients started on 1 μg/kg of romiplostim, including 15 (23%) patients who had been on placebo in the previous study. All 65 patients received their doses per protocol ≥90% of the time; 21 patients missed ≥1 dose for noncompliance a total of 65 times. Dose over time was typically around 4-5 μg/kg, as shown in FIG. 3A. After week 240 (n≤7), the dose fluctuated.

### Safety

The most common adverse events were headache and contusion (Table 3, infra). Fifty-four serious adverse events occurred in 19 patients but were considered treatment-related in only 1 patient who had concurrent grade 4 thrombocytopenia, grade 3 epistaxis, and grade 2 anemia (full list of serious adverse events in Supp. Table 1, infra). Bleeding adverse events occurred in 57 patients; 3 bleeding adverse events were deemed treatment related (injection site hemorrhage, injection site bruising, and epistaxis). The most frequent bleeding adverse events were contusion (51%), epistaxis (49%), petechiae (31%), and gingival bleeding (20%). There were no cases of intracranial hemorrhage; specific bleeding events reported included menorrhagia (n=7, 4%), hematuria (n=4, 2%), rectal hemorrhage (n=4, 2%), hemoptysis (n=3, 2%), anal hemorrhage (n=2, 1%), hematochezia (n=2, 1%), and hematemesis (n=1, 0.6%). There were 7 patients with serious or grade 3 bleeding (Supp. Table 2, infra); 1 case of worsening epistaxis was considered treatment-related. No arterial or venous thromboembolic events were reported. Of note, the contusion rate would drop from 239 to 92 per 100 patient-years if one patient who had 499 adverse events is excluded. This child, a 7-year-old boy who was in the study for 3.4 years, also had several serious adverse events: 6 of platelet count decreased, and 1 each of headache, head injury, vomiting, leukopenia, hematoma, pharyngitis streptococcal, and gastroenteritis.

Post-dosing antibodies were assayed annually in 60 patients. One patient had anti-romiplostim neutralizing antibody detected upon leaving the study to receive other therapy; the neutralizing antibody was absent on retesting 3 and 6 months later. She required multiple additional therapies and has been stable on mycophenolate. No patients had anti-TPO neutralizing antibody, despite yearly antibody assays covering over 200 patient-years of exposure (which includes time on previous romiplostim studies).

Bone marrow biopsies were performed in 2 patients with additional cytopenias; both patients were found to have iron-deficiency anemia and no fibrosis or malignancy. One biopsy, performed after 2 years on study was in a 17-year-old girl to evaluate her persistent anemia. With regular supplemental iron intake and lighter menstrual bleeding, her anemia improved. The second biopsy was performed after 6 weeks on study in an 11-year-old girl as she developed neutropenia and anemia; she received iron for the anemia and had intermittent neutropenia which eventually resolved.

### Efficacy

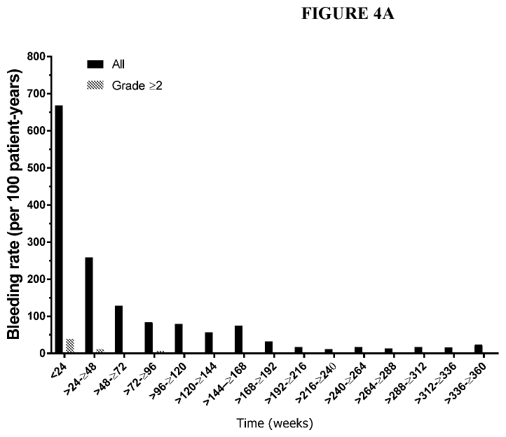

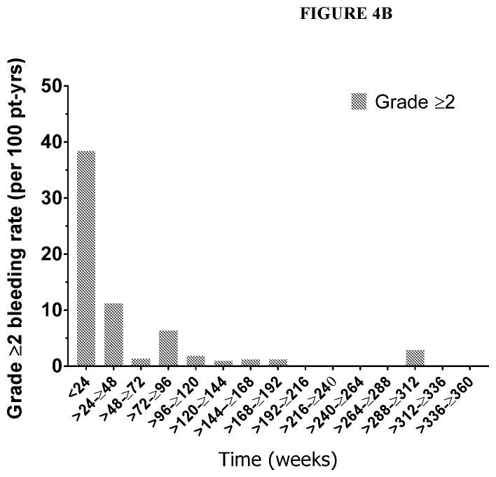

From week 2 on, median platelet counts remained >50×10⁹/L; median platelet counts were >100×10⁹/L from week 24 to week 260 (FIG. 3B). Nearly all (94%, 61/65) patients had ≥1 platelet response (platelet counts ≥50×10⁹/L, excluding counts ≤4 weeks after rescue medication). Most (72%, 47/65) patients had a platelet response ≥75% of the time and over half (58%, 38/65) had a platelet response ≥90% of the time. Sixty (92%) patients (or caregivers) self-administered romiplostim (i.e., at home, not in the clinic). Twenty-three (35%) patients received rescue medications (Table 4, infra); usage was highest in the first few months. At baseline, 5 patients were taking other ITP medications such as aminocaproic acid, prednisolone, prednisone, and tranexamic acid). Overall, 48% (31/65) of patients were taking other ITP medications (either from baseline or as rescue); the rate of patients taking these medications decreased over the course of the study. Bleeding, both overall and grade ≥2, decreased over time (FIGS. 4A and B).

### Remission/Treatment-Free Periods

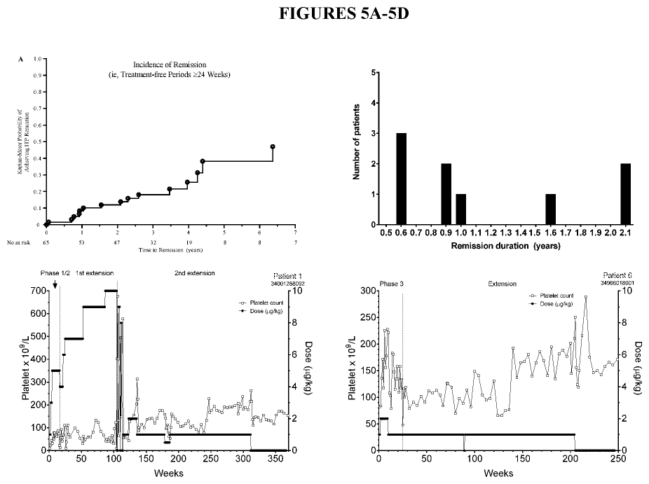

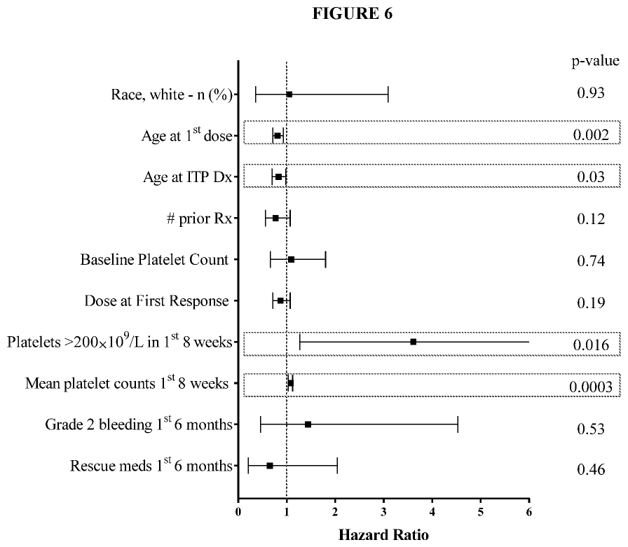

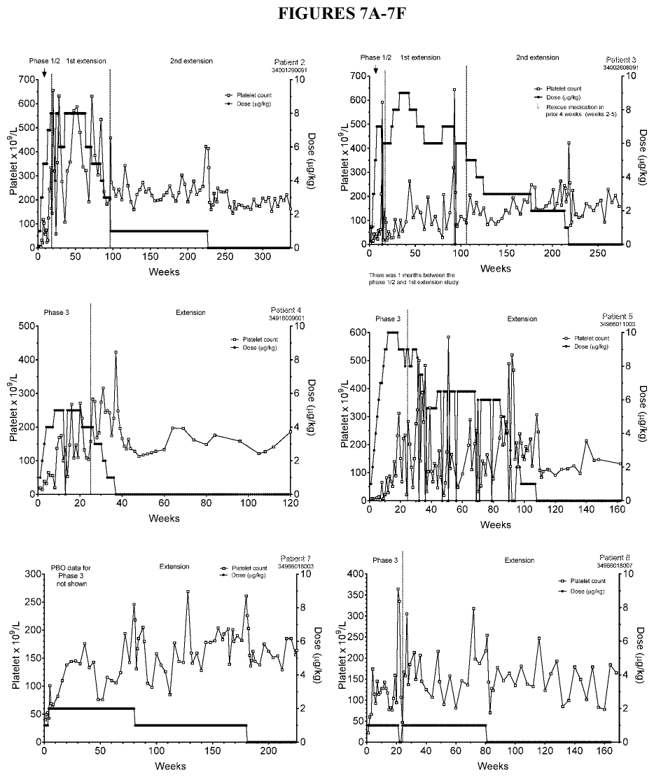

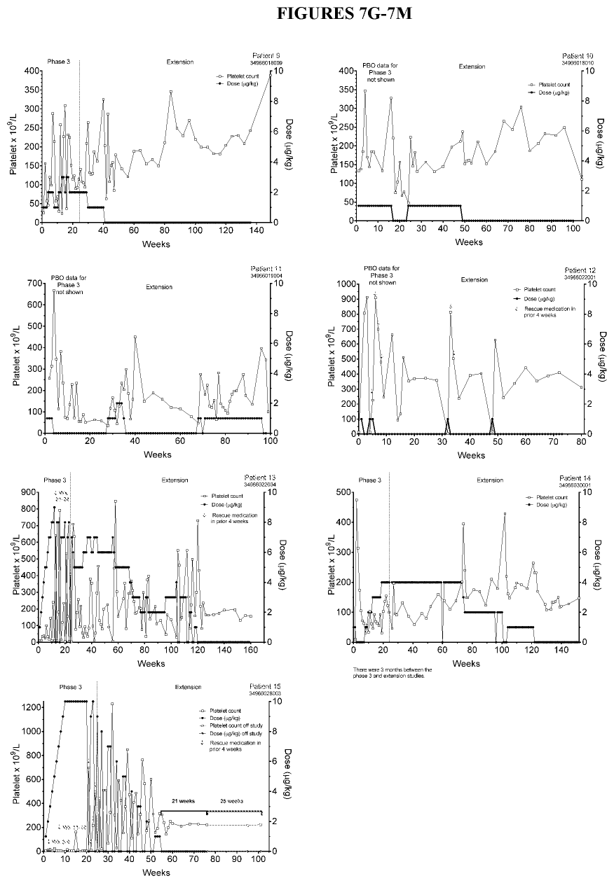

Fifteen (23%) patients maintained platelet counts ≥50×10⁹/L for ≥24 weeks with no ITP medications (i.e., a treatment-free period, here defined as remission; Table 5, FIGS. 5A-D). At the start of the treatment-free periods, these patients (9 girls, 6 boys) had had ITP for a median (min-max) of 4 (1-12) years and had received romiplostim for 3 (1-7) years. None of these patients had had prior splenectomy. Median (min-max) baseline platelet counts were 14 (1-44)×10⁹/L. These treatment-free periods lasted for a median (min-max) of 1 (0.6-2.1) years, with all but one patient still off all treatments with platelet counts ≥50×10⁹/L at study end. In the last few months of reducing the dose, the median (min-max) peak platelet counts were 299 (217-730)×10⁹/L. After the first month of discontinuation, when drug effect/rebound would no longer be expected, the median (min-max) peak platelet counts were 249 (150-450)×10⁹/L and the median (min-max) lowest platelet counts were 113 (77-311)×10⁹/L (as is visible in individual patient plots, FIGS. 5A-D). All 15 of these patients had platelet counts over 100×10⁹/L for ≥3 months and 12/15 for ≥6 months, all with no ITP medications. The median (min-max) duration of being ≥100×10⁹/L for these 15 patients was 42 (13-109) weeks.

Baseline characteristics and early treatment outcomes such as ITP duration, past ITP treatments, and platelet counts in the first 4 weeks were evaluated in post hoc analyses. Younger age at diagnosis, younger age at first dose, platelets to ≥200×10⁹/L in the first 4 weeks, and higher mean platelet count in the first 4 weeks were all associated with a greater likelihood of developing remission in univariate analyses (Table 5, FIG. 3). In multivariate analyses, age at first dose (p=0.001) and platelet counts to ≥200×10⁹/L in the first 4 weeks (p=0.004) were predictive of remission. Of note, the models for prior rituximab use (p=1.0) and prior splenectomy (p=0.32) had non-proportional hazards, hence no hazard ratio. When multivariate analyses were done by baseline age, for those <10 years of age at first dose (N=32), <3 prior ITP treatments and platelet counts to ≥200×10⁹/L in the first 12 weeks were predictive of remission; for those <10 years of age at diagnosis (N=49), younger age at first dose, platelet counts to ≥200×10⁹/L in the first 4 weeks, mean platelet count ≥100×10⁹/L in the first 12 weeks, and use of rescue medication in the first 6 months were also predictive, with rescue medication use being a negative predictor. There were too few patients with age ≥10 years to model for factors predictive of remission.

### Discussion

### Efficacy

Romiplostim treatment was frequently associated with sustained platelet response, as shown by the findings that most (72%) children had a platelet response ≥75% of the time and over half (58%, 38/65) had a platelet response ≥90% of the time. Further, median platelet counts were maintained in the desired range (50-200×10⁹/L) from week 2 on and were >100×10⁹/L from week 24 to week 260.

The patients in this study were also able to decrease use of other ITP medications and reported less bleeding over time, particularly clinically meaningful bleeding (grade ≥2).

### Safety

Data from this study showed that romiplostim was well tolerated, with no thrombotic events, bone marrow changes, fatalities, or new safety concerns, despite 182 patient-years of exposure to romiplostim; i.e., nearly 3 years per patient.

Median dose was the same as in the phase 3 study (˜4-5 μg/kg).

About 30% of patients had serious adverse events, but only one patient had treatment-related serious adverse events (thrombocytopenia, epistaxis, and anemia).

Despite the large number of patient-years of treatment, only one patient was identified to develop neutralizing anti-romiplostim antibodies, and no patients had neutralizing antibody to TPO. This is consistent with what has been observed in adults treated with ITP; in an integrated database of romiplostim ITP trials, anti-romiplostim neutralizing antibodies were found in 4 out of 1046 patients with a total exposure of 1832 patient-years.⁹ All 4 patients continued to have platelet responses with romiplostim.

Bone marrow biopsies were performed as the investigators thought clinically indicated. Two patients had bone marrow biopsies performed as they had additional cytopenias; both had iron-deficient anemia.

No cases of thrombosis were seen. In adult studies of romiplostim, rates of thrombosis have been low and not different from placebo/standard of care (Cines et al., 2015).¹⁰

While only two patients discontinued due to adverse events, overall, 42% (28/66) of patients left before study end, with the most common reasons being withdrawal of consent (n=10) and required alternative therapy (n=4).

### Remission/Treatment-Free Periods

A pleasant surprise was that nearly one quarter (15/65) patients, who were able to discontinue all ITP treatments (including romiplostim) after receiving 0.7-6 years of romiplostim and still maintain hemostatic platelet counts (≥50×10⁹/L) for at least 6 months, which we defined here as remission. Only 1/15 patients relapsed and needed further treatment of 1 μg/kg romiplostim. At last contact, this patient had once more discontinued romiplostim as platelet counts had reached 400×10⁹/L.

In a multivariate analysis, younger age at first dose and higher mean platelet count in the first 8 weeks were both associated with a greater likelihood of developing remission. This is of particular interest as previously it was observed in a study of adults that higher mean platelet count in the first 8 weeks was also associated with increased likelihood of developing remission, although the authors ultimately concluded that no statistically significant predictors of remission were found when examining other demographic or baseline characteristics. (Newland et al., 2016).

Regarding remission analyses, we should point out that even if factors were not found to be predictive of remission in this study, that could be as the populations were small or not the right population to study it. Absence of evidence is not evidence of absence.

Further, it should be noted that the definition for remission can vary significantly, in some cases having higher platelet thresholds (e.g. 100×10⁹/L instead of 50×10⁹/L) and/or longer durations (e.g., one year instead of 6 months). For purposes of this specification, remission is defined as maintaining a platelet threshould of 50×10⁹/L for at least 6 months (24 weeks) with no ITP medications.

Development of remission was not wholly expected, and remission was not a predetermined study endpoint, but a phenomenon that was observed.

While remission in children is likely very different than that observed in adults, this finding is consistent with past romiplostim studies in adults with ITP, including one in which 75 patients who had ITP for ≤6 months, were treated with romiplostim for ≤12 months and then had a forced taper (Newland et al., 2016).¹¹ Remission was observed in 24 patients (32%), with no significant predictors. Most (20/24) patients had remission start before the forced taper.

Treatment-free periods were also seen in adults with ITP in the eltrombopag extension study (EXTEND), (Saleh et al., 2013)¹² in which a prolonged response (defined as platelets counts ≥50×10⁹/L for ≥12 weeks with no ITP medications, including eltrombopag) was found in 13 of 325 patients (4%). The median (min-max) time since ITP diagnosis was 26 months (9-128) and the median (min-max) duration of eltrombopag treatment in the extension before the prolonged response was 160 days (14-1107).

In a retrospective evaluation of adults with ITP receiving eltrombopag, eltombopag was discontinued in 80/201 patients with complete response (platelet counts >100×10⁹/L) (Gonzalez-Lopez et al., 2015)¹³. Of 49 evaluable patients, 26 showed sustained response for at least 6 months after discontinuing eltrombopag without additional ITP therapy. No predictive factors of sustained response after eltrombopag withdrawal were identified.

As noted above, definitions of response, remission, and sustained response can vary considerably. In the study reported herein, we chose platelet counts ≥50×10⁹/L for response and platelet counts ≥50×10⁹/L for ≥6 months with no ITP medications for remission. Other studies have used different platelet thresholds for response and treatment-free periods, such as response per IWG (Rodeghiero et al.)¹⁴, in which thresholds of 30×10⁹/L and 100×10⁹/L were used for response and complete response, both in the absence of bleeding, or treatment-free periods of at least a year, as in a long-term rituximab study (Patel et al., 2012).¹⁵

Longer ITP duration before entering remission, as seen in our study, indicates that this remission is likely not spontaneous, but due to romiplostim and suggests that romiplostim could be disease-altering, which is not expected as romiplostim is not known to be immunomodulatory. While intending not to be bound by theory, possible mechanisms of action include:
Enhanced T-regulatory cell function (Bao et al., 2010; Chong, 2010; Son et al., 2015)¹⁶⁻¹⁸
Natural killer T cells (Johansson et al., 2005)¹⁹
B-regulatory cell activity (Li et al., 2012)²⁰
Induction of fragment crystallizable receptor IIb (FcRIIb), the inhibitory FcR (Liu et al., 2016)²¹

Development of remission in almost 1/4 of patients suggests that maintenance with romiplostim may not be an indefinite “life-long treatment”; response in the first 8 weeks may indicate whether a patient is more likely to enter remission.

TABLE 1
Baseline Demographics

| | N = 66 | |
|---|---|---|
| | n (%) | |
| Female | 37 | (56) |
| Race/Ethnicity | | |
| White | 40 | (61) |
| African American | 9 | (14) |
| Hispanic/Latino | 9 | (14) |
| Asian | 6 | (9) |
| Other | 2 | (3) |
| Median age (min, max), years | 11 | (3, 18) |
| Age group, years | | |
| ≥1-<6 | 12 | (18) |
| ≥6-<12 | 25 | (38) |
| ≥12 | 29 | (44) |
| Median baseline platelet count × 10⁹/L (min, max) * | 28 | (2, 458) |

* For extension study described in this paper (i.e., not parent studies)

TABLE 2
Patient ITP Medication History

| | N = 66 | |
|---|---|---|
| | n (%) | |
| ITP duration, median (min-max), years | 3.0 | (1.7-5.6) |
| Number of prior ITP treatments | | |
| 1 | 7 | (11) |
| 2 | 17 | (26) |
| 3 | 15 | (23) |
| >3 | 26 | (39) |
| Prior splenectomy | 6 | (9) |
| Number of patients receiving specific | | |
| therapies in the past | | |
| IVIg | 60 | (91) |
| Corticosteroid | 54 | (82) |
| Anti-D Antibody (WinRho) | 24 | (36) |
| Rituximab | 24 | (36) |
| Vincristine/Vinblastine | 4 | (6) |
| Danazol | 4 | (6) |
| Azathioprine | 4 | (6) |
| Other* | 26 | (39) |

*Other includes aminocaproic acid, cyclosporine, dapsone, mercaptopurine, mycophenolate, platelets, sirolimus, and tranexamic acid. Please note that designation of platelet transfusions as a rescue medication was per investigator.

TABLE 3
Adverse Events

| | | | | Duration-adjusted Events | | | |
|---|---|---|---|---|---|---|---|
| | | | | (per 100 pt-yr) | | | |
| | | | | | | Excluding Pt | |
| | Adverse | N = 65 | | All | | with 499 AEs | |
| Category | Event | n (%) | | 182 pt-yr | | 179 pt-yr | |
| Most | Headache | 38 | (59) | 151 | (83) | 126 | (71) |
| common | Contusion | 33 | (51) | 435 | (239) | 164 | (92) |
| adverse | Epistaxis | 32 | (49) | 103 | (57) | 98 | (55) |
| events | Upper | 32 | (49) | 101 | (56) | 101 | (57) |
| | respiratory | | | | | | |
| | tract | | | | | | |
| | infection | | | | | | |
| Serious | Any | 19 | (29) | 54 | (30) | 41 | (23) |
| adverse | Thrombo- | 4 | (6) | 6 | (3) | 6 | (3) |
| events* | cytopenia | | | | | | |
| | Pyrexia | 3 | (5) | 3 | (2) | 3 | (2) |
| | Epistaxis | 2 | (3) | 2 | (1) | 2 | (1) |
| | Headache | 2 | (3) | 2 | (1) | 1 | (0.6) |
| | Vomiting | 2 | (3) | 2 | (1) | 1 | (0.6) |

*Most common serious adverse events included; a full list is in supplemental tables.

TABLE 4
Rescue Medications

| | |
|---|---|
| N = 65 | Events |
| n (%) | (rate per 100 pt-yr) |

| | | | | |
|---|---|---|---|---|
| Any use | 23 | (35) | 80 | (44) |
| Intravenous | 10 | (15) | 31 | (17) |
| immunoglobulin | | | | |
| Corticosteroids* | 13 | (20) | 31 | (17) |
| Antifibrinolytic | 6 | (9) | 14 | (8) |
| (aminocaproic | | | | |
| acid or | | | | |
| tranexamic acid) | | | | |
| Azathioprine | 1 | (2) | 1 | (0.6) |
| Red blood cell | 1 | (2) | 1 | (0.6) |
| transfusion | | | | |
| Platelet | 1 | (2) | 2 | (1) |
| transfusion | | | | |

*Corticosteroids include prednisone/prednisolone, methylprednisolone, dexamethasone

TABLE 5
Treatment-free periods of ≥24 weeks with platelet counts ≤50 × 10⁹/L (here defined as remission)

| Patient number | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| Age at remission | 16 | 6 | 10 | 8 | 6 | 12 | 18 | 7 | 6 | 9 | 4 | 5 | 6 | 14 | 16 |
| start, years | | | | | | | | | | | | | | | |
| Sex | F | M | M | F | F | F | F | M | M | F | M | F | F | M | F |
| Race/Ethnicity | W | B | W | W | W | W | W | W | B | A | W | B | H | W | W |
| ITP, years^{‡} | 7 | 6 | 5 | 1 | 4 | 11 | 12 | 3 | 1 | 2 | 3 | 4 | 3 | 3 | 5 |
| # past ITP | 3 | 6 | 4 | 2 | 2 | 3 | 4 | 5 | 2 | 1 | 2 | 1 | 4 | 3 | 4 |
| therapies (prior | | | | | | | | | | | | | | | |
| to studies) | | | | | | | | | | | | | | | |
| Past rituximab | 4 | 5 | 5 | N | N | N | N | N | N | N | N | N | 2 | N | 5 |
| use, years prior | | | | | | | | | | | | | | | |
| to remission start | | | | | | | | | | | | | | | |
| Baseline platelet | 12 | 5 | 9 | 18 | 7 | 44 | 15 | 26 | 28 | 28 | 25 | 1 | 11 | 14 | 4 |
| count (×10⁹/L)^{τ} | | | | | | | | | | | | | | | |
| Romiplostim, | 7 | 6 | 5 | 2 | 3 | 5 | 4 | 3 | 3 | 2 | 2 | 2 | 3 | 1 | 3 |
| years^{‡} | | | | | | | | | | | | | | | |
| Maximum dose, | 10 | 8 | 9 | 5 | 10 | 2 | 2 | 1 | 3 | 1 | 2 | 1 | 9 | 10 | 4 |
| μg/kg | | | | | | | | | | | | | | | |
| ITP remission, | 1.1 | 2.1 | 1.1 | 1.6 | 1.0 | 0.8 | 0.9 | 1.7 | 2.1 | 1.1 | 0.6* | 0.6 | 0.8 | 0.4^{†} | 0.6 |
| years | | | | | | | | | | | | | | | |

Data are integrated over parent study and extension study.
A, Asian; B, Black; F, female; H, Hispanic/Latino; M, male; N, no rituximab use; Rx, therapy; W, white.
^{τ}At start of parent study (not extension).
^{‡}At remission start.
*Remission ended before study end.
^{†}This patient met remission criteria for 0.4 years on study and ≥0.5 years post study.

TABLE 6
Predictors of remission

| | Patients | | Patients | | | | |
|---|---|---|---|---|---|---|---|
| | with | | without | | | | |
| | Remission | | Remission | | | HR 95% | |
| Characteristic | (N = 15) | | (N = 50) | | HR | CI | P-value |
| Sex, female, n (%) | 9 | (60) | 27 | (54) | 1.19 | (0.42, 3.41) | 0.74 |
| Race, white, n (%) | 10 | (67) | 30 | (60) | 1.05 | (0.36, 3.09) | 0.93 |
| Age at 1^{st} dose | 6.5 | (4.0) | 10.6 | (4.0) | 0.81 | (0.71, 0.93) | 0.002 |
| Age at ITP Dx | 4.8 | (3.6) | 7.5 | (3.4) | 0.83 | (0.70, 0.98) | 0.03 |
| Baseline ITP duration | 2.3 | (2.4) | 3.6 | (2.7) | 0.79 | (0.61, 1.03) | 0.08 |
| Baseline platelet count* | 16.5 | (11.8) | 15.9 | (9.5) | 1.09 | (0.66, 1.80) | 0.74 |
| # prior Rx | 3.1 | (1.4) | 3.3 | (1.9) | 0.77 | (0.56, 1.07) | 0.12 |
| Prior rituximab, n (%) | 5 | (33) | 19 | (38) | N/A | N/A | 1 |
| Splenectomized, n (%) | 0 | (0) | 6 | (12) | N/A | N/A | 0.32 |
| Dose at first response (μg/kg) | 3.1 | (2.9) | 4.1 | (3.0) | 0.87 | (0.71, 1.07) | 0.19 |
| Platelet count >200 × 10⁹/L | 4 | (27) | 3 | (6) | 5.48 | (1.63, 18.42) | 0.006 |
| in 1^{st} 4 weeks | | | | | | | |
| Mean platelet counts 1^{st} 4 weeks* | 128 | (149) | 57.5 | (42.9) | 1.09 | (1.04, 1.13) | <0.0001 |
| Grade ≥2 bleeding 1^{st} 6 months | 4 | (27) | 8 | (16) | 1.44 | (0.46, 4.53) | 0.53 |
| Rescue meds 1^{st} 6 months | 4 | (27) | 20 | (40) | 0.65 | (0.21, 2.04) | 0.46 |

Data are median (min-max) or mean (SD) unless indicated otherwise. HR, hazard ratio; w/o, without.
*= per 10 × 10⁹/L.
N/A for HR and 95% CI when proportional hazards assumption in model was violated and model results are not reliable.
P-value calculated using Fisher's exact test (categorical variables) or Kruskal-Wallis test (continuous variables).

### Supplemental Tables

SUPP. TABLE 1
Serious Adverse Events

| | N = 65 |
|---|---|
| | n (%) |
| Thrombocytopenia | 4 (6) |
| Pyrexia | 3 (5) |
| Epistaxis | 2 (3) |
| Headache | 2 (3) |
| Vomiting | 2 (3) |
| Anemia | 1 (2) |
| Asthenia | 1 (2) |
| Asthma | 1 (2) |
| Biliary dyskinesia | 1 (2) |
| Clostridium difficile infection | 1 (2) |
| Contusion | 1 (2) |
| Dehydration | 1 (2) |
| Depression | 1 (2) |
| Febrile neutropenia | 1 (2) |
| Gastroenteritis | 1 (2) |
| Gastrointestinal infection | 1 (2) |
| Gingivitis | 1 (2) |
| Hemangioma | 1 (2) |
| Hematoma | 1 (2) |
| Head injury | 1 (2) |
| ITP | 1 (2) |
| Infection | 1 (2) |
| Leukopenia | 1 (2) |
| Meningitis viral | 1 (2) |
| Metapneumovirus infection | 1 (2) |
| Mouth hemorrhage | 1 (2) |
| Pharyngitis streptococcal | 1 (2) |
| Platelet count decreased | 1 (2) |
| Pneumonia mycoplasmal | 1 (2) |
| Post procedural hemorrhage | 1 (2) |
| Respiratory syncytial virus infection | 1 (2) |
| Subcutaneous abscess | 1 (2) |
| Suicidal ideation | 1 (2) |
| Transfusion reaction | 1 (2) |
| Ulcer hemorrhage | 1 (2) |
| Viral infection | 1 (2) |
| Viral upper respiratory tract infection | 1 (2) |

Supp. Table 2
Serious and/or Grade 3 Bleeding Adverse Events

| | | Age | | | | Duration | | | | |
|---|---|---|---|---|---|---|---|---|---|---|
| | | (y) | Sex | Week | Bleeding Adverse Events | (days) | Grade | Related | Serious | Actions |
| | Serious | 7 | Boy* | 96 | Hematoma | 16 | 1 | N | Y | Hospitalized, no |
| | bleeding | | | | | | | | | change romiplostim |
| | | 4 | Boy | 24 | Epistaxis and mouth | 1 | 2 | N | Y | Hospitalized, no |
| | | | | | hemorrhage | | | | | change romiplostim |
| | | | | 86 | Hemorrhage after | 1 | 2 | N | Y | Hospitalized, no |
| | | | | | tonsillectomy (platelet | | | | | change romiplostim |
| | | | | | count day before was | | | | | |
| | | | | | 191 × 10⁹/L | | | | | |
| | | 11 | Girl | 40 | Worsening ITP and | 3 | 1 | N | Y | Hospitalized, left the |
| | | | | | contusion | | | | | study a few days later |
| | | | | | | | | | | for noncompliance |
| Grade 3 | | 7 | Boy | 12 | Worsening epistaxis, | 2 | 3 | Y | Y | Hospitalized, no |
| bleeding | | | | | concurrent grade 4 | | | | | change romiplostim |
| | | | | | thrombocytopenia | | | | | (later D/C as req |
| | | | | | | | | | | other Rx) |
| | | 3 | Girl | 3 | Bleeding mouth sores | 1 | 3 | N | Y | ER |
| | | 14 | Boy | 295 | Hematuria | 5 | 3 | N | N | ER, no change |
| | | | | | | | | | | romiplostim |
| | | 8 | Boy | 94 | Increased petechiae | 13 | 3 | N | N | No change |
| | | | | | | | | | | romiplostim |
| | | | | | | | | | | (later D/C as req |
| | | | | | | | | | | other Rx) |

*This patient reported 499 adverse events.
ER, emergency room; N, no; Y, yes.

Retrospective statistical analyses were performed concerning two adult studies (clinical trials 20080009 and 20080435) to confirm that the results seen with the above-described pediatric study correlated with the adult population. The '0009 trial, reported in Janssens et al. (2016), concerned chronic ITP, with romiplostim treatment up to three years. The '0435 trial, reported in Newland et al. (2015), concerned early ITP treatment with romiplostim for up to one year, with a specific attempt to taper to see if the subject would go into remission. Both studies individually showed that having a platelet count ≥200×10⁹/L within the first four, eight, and twelve weeks of treatment with romiplostim is associated a higher rate of remission. For the '0009 study, a secondary, more conservative test of association was used. For both adult studies, reaching a platelet count ≥200×10⁹/L within the first eight weeks is the strongest predictor of entering remission. The results of the analyses appear in Tables A through D below.

TABLE A
Univariate Analysis regarding Clinical Trial 20080009

| | Patient | Patients | | | |
|---|---|---|---|---|---|
| | with | without | | | |
| | Remission | Remission | Hazard | 95% CI | |
| Variable | (N = 24) | (N = 51) | Ratio | for HR | p-value |
| Platelets | 9 (38) | 11 (22) | 2.30 | (1.00, 5.27) | 0.0491 |
| >200 in | | | | | |
| first 4 | | | | | |
| weeks, | | | | | |
| Yes - n | | | | | |
| (%) | | | | | |
| Platelets | 15 (63) | 18 (35) | 2.83 | (1.23, 6.49) | 0.0140 |
| >200 in | | | | | |
| first 8 | | | | | |
| weeks, | | | | | |
| Yes - n | | | | | |
| (%) | | | | | |
| Platelets | 16 (67) | 24 (47) | 2.14 | (0.91, 5.00) | 0.0806 |
| >200 in | | | | | |
| first 12 | | | | | |
| weeks, | | | | | |
| Yes - n | | | | | |
| (%) | | | | | |

Hazard ratio (HR), 95% confidence interval (CI) and p-value calculated using a proportional hazards model with a single explanatory variable. An HR >1 indicates a higher likelihood for remission. N/A for HR and 95% CI when proportional hazards assumption in model was violated and model results are not reliable. P-value calculated using Fisher's exact test to check for association.

TABLE B
Remitting Patients with Platelet Count ≥ 200 ×
10⁹/L at Week 4, 8, and 12 in Clinical Trial 20080009

| | With | With |
|---|---|---|
| | Platelets | Platelets |
| | ≥200 | <200 |
| n/N with remission (%) - Week 4 | 9/20 (45%) | 15/55 (27%) |
| n/N with remission (%) - Week 8 | 15/33 (45%) | 9/42 (21%) |
| n/N with remission (%) - Week 12 | 16/40 (40%) | 8/35 (23%) |

TABLE C
Univariate Analysis regarding Clinical Trial 20080435

| | Patient | Patients | | | |
|---|---|---|---|---|---|
| | with | without | | | |
| | Remission | Remission | Hazard | 95% CI | |
| Variable | (N = 25) | (N = 144) | Ratio | for HR | p-value |
| Platelets | 9 (36) | 25 (17) | 2.54 | (1.12, 5.74) | 0.0257 |
| ≥200 in | | | | | |
| first 4 | | | | | |
| weeks, | | | | | |
| Yes - n (%) | | | | | |
| Platelets | 16 (64) | 45 (31) | N/A | N/A | 0.0028 |
| ≥200 in | | | | | |
| first 8 | | | | | |
| weeks, | | | | | |
| Yes - n (%) | | | | | |
| Platelets | 17 (68) | 55 (38) | N/A | N/A | 0.0079 |
| ≥200 in | | | | | |
| first 12 | | | | | |
| weeks, | | | | | |
| Yes - n (%) | | | | | |

Hazard ratio (HR), 95% confidence interval (CI) and p-value were calculated using a proportional hazards model with a single explanatory variable. An HR >1 indicates a higher likelihood for remission. N/A for HR and 95% CI when proportional hazards assumption in model was violated and model results are not reliable. P-value calculated using Fisher's exact test to check for association.

TABLE D
Remitting Patients with Platelet Count ≥ 200 ×
10⁹/L at Week 4, 8, and 12 in Clinical Trial 20080435

| | With | With |
|---|---|---|
| | Platelets | Platelets |
| | ≥200 | <200 |
| n/N with remission (%) - Week 4 | 9/20 (45%) | 15/55 (27%) |
| n/N with remission (%) - Week 8 | 15/33 (45%) | 9/42 (21%) |
| n/N with remission (%) - Week 12 | 16/40 (40%) | 8/35 (23%) |

### REFERENCES

Bao W, Bussel J B, Heck S, et al. (2010). Improved regulatory T-cell activity in patients with chronic immune thrombocytopenia treated with thrombopoietic agents. Blood 116: 4639-45.
Bussel J. B., Hsieh L, Buchanan G R, et al. (2014). Long-term use of the thrombopoietin-mimetic romiplostim in children with severe chronic immune thrombocytopenia (ITP). Pediatr Blood Cancer 62: 208-13.
Bussel J. B., Buchanan G R, Nugent D J, et al. (2011). A randomized, double-blind study of romiplostim to determine its safety and efficacy in children with immune thrombocytopenia. Blood 118: 28-36.
Bussel, J. B., Kuter, D. J., Pullarkat, V., Lyons, R. M., Guo, M. & Nichol, J. L. (2009).
Safety and efficacy of long-term treatment with romiplostim in thrombocytopenic patients with chronic ITP. Blood, 113, 2161-2171.
Chong B. H. (2010). ITP: Tregs come to the rescue. Blood 116: 4388-90.
Cines D. B., Gernsheimer T, Wasser J, et al. (2015). Integrated analysis of long-term safety in patients with chronic immune thrombocytopaenia (ITP) treated with the thrombopoietin (TPO) receptor agonist romiplostim. Int J Hematol 102: 259-70.
Cines D. B., Wasser J, Rodeghiero F, et al. (2017). Safety and efficacy of romiplostim in splenectomized and nonsplenectomized patients with primary immune thrombocytopenia. Haematologica 102: 1342-51.
George J. N., Woolf S. H., Raskob G. E., et al. (1996). Idiopathic thrombocytopenic purpura: a practice guideline developed by explicit methods for the American Society of Hematology. Blood 88: 3-40.
Ghanima, W., Godeau, B., Cines, D. B. & Bussel, J. B. (2012). How I treat immune thrombocytopenia: the choice between splenectomy or a medical therapy as a second-line treatment. Blood, 120, 960-969.
Gonzalez-Lopez, T. J., Pascual, C., Alvarez-Roman M. T. et al. (2015). Successful discontinuation of eltrombopag after complete remission in patients with primary immune thrombocytopenia. Am J Hematol 90: E40-3.
Janssens A., Rodeghiero F., Anderson D., Chong B. H., Boda Z., Pabinger I., Červinek L., Terrell D. R., Wang X., Franklin J. (2016). Changes in bone marrow morphology in adults receiving romiplostim for the treatment of thrombocytopenia associated with primary immune thrombocytopenia. Ann Hematol. June; 95(7):1077-87. doi: 10.1007/s00277-016-2682-2. Epub 2016 Apr. 30. PubMed PMID: 27130310; PubMed Central PMCID: PMC4889627.
Kuter, D. J., Bussel, J. B., Lyons, R. M., Pullarkat, V., Gernsheimer, T. B., Senecal, F. M., Aledort, L. M., George, J. N., Kessler, C. M., Sanz, M. A., Liebman, H. A., Slovick, F. T., de Wolf, J. T., Bourgeois, E., Guthrie, T. H. Jr, Newland, A., Wasser, J. S., Hamburg, S. I., Grande, C., Lefrere, F., Lichtin, A. E., Tarantino, M. D., Terebelo, H. R., Viallard, J. F., Cuevas, F. J., Go, R. S., Henry, D. H., Redner, R. L., Rice, L., Schipperus, M. R., Guo, D. M. & Nichol, J. L. (2008). Efficacy of romiplostim in patients with chronic immune thrombocytopenic purpura: a double-blind randomised controlled trial. Lancet, 371, 395-403.
Kuter, D. J., Bussel, J. B., Newland, A., Baker, R I., Lyons, R. M., Wasser, J., Viallard, J. F., Macik, G., Rummel, M., Nie, K. & Jun, S. (2013). Longterm treatment with romiplostim in patients with chronic immune thrombocytopenia: safety and efficacy. British Journal of Haematology, 161, 411-423.
Kuter, D. J., Rummel, M., Boccia, R., Macik, B. G., Pabinger, I., Selleslag, D., Rodeghiero, F., Chong, B. H., Wang, X. & Berger, D. P. (2010) Romiplostim or standard of care in patients with immune thrombocytopenia. New England Journal of Medicine, 363, 1889-1899.
Li X., Zhong H., Bao W. et al. (2012). Defective regulatory B-cell compartment in patients with immune thrombocytopenia. Blood 120: 3318-25.
Liu X. G., Liu S., Feng Q. et al. (2016). Thrombopoietin receptor agonists shift the balance of Fcgamma receptors toward inhibitory receptor IIb on monocytes in ITP. Blood 128: 852-61.
Mahevas, M., Fain, O., Ebbo, M., Roudot-Thoraval, F., Limal, N., Khellaf, M., Schleinitz, N., Bierling, P., Godeau, B. & Michel, M. (2014). The temporary use of thrombopoietin-receptor agonists may induce a prolonged remission in adult chronic immune thrombocytopenia. Results of a French observational study. British Journal of Haematology, 165, 865-869.
Newland A., Godeau B., Priego V. et al. (2016). Remission and platelet responses with romiplostim in primary immune thrombocytopenia: final results from a phase 2 study. Br J Haematol 172: 262-73. doi: 10.1111/bjh.13827. Epub 2015 Nov. 5. PubMed PMID: 26537623.
Nugent D., McMillan R., Nichol J. L., Slichter S. J. (2009). Pathogenesis of chronic immune thrombocytopenia: increased platelet destruction and/or decreased platelet production. Br J Haematol 146: 585-96.
Oved J., Lee C., Bussel J. (2017). Treatment of Children with Persistent and Chronic Idiopathic Thrombocytopenic Purpura: 4 Infusions of Rituximab and Three 4-Day Cycles of Dexamethasone. Journal of Pediatrics (in press).
Patel V. L., Mahevas M., Lee S. Y. et al. (2012). Outcomes 5 years after response to rituximab therapy in children and adults with immune thrombocytopenia. Blood 119: 5989-95.
Provan D., Stasi R., Newland A. C. et al. (2010). International consensus report on the investigation and management of primary immune thrombocytopenia. Blood 115: 168-86.
Provan, D., Taylor, L., Nandigham, R., Doobaree, U., Kalkur, P. & Newland, A. (2014). Sustained responses following treatment with romiplostim in immune thrombocytopenia: a single-centre experience. Journal of Hematology & Thromboembolic Diseases, 2, 147
Rodeghiero F., Stasi R., Gernsheimer T. et al. (2009). Standardization of terminology, definitions and outcome criteria in immune thrombocytopenic purpura of adults and children: report from an international working group. Blood 113: 2386-93.
Saleh M. N., Bussel J. B., Cheng G. et al. (2013). Safety and efficacy of eltrombopag for treatment of chronic immune thrombocytopenia: results of the long-term, open-label EXTEND study. Blood 121: 537-45.
Son B. R., Kim J. Y. (2015). Association of CD4(+)CD25(+)FoxP3(+) regulatory T cells with natural course of childhood chronic immune thrombocytopenic purpura. Korean J Pediatr 58: 178-82.
Tarantino et al, ASH 2017, abstract 14 as at: https://ash.confex.com/ash/2017/webprogram/Paper100126.html
Tarantino M. D., Bussel J. B., Blanchette V. S. et al. (2016). Romiplostim in children with immune thrombocytopenia: a phase 3, randomised, double-blind, placebo-controlled study. Lancet 388: 45-54.
Thachil, J., Salter, I., George, J. N. (2013). Complete remission of refractory immune thrombocytopenia (ITP) with a short course of Romiplostim. European Journal of Haematology, 91, 376-377.
Wang B., Nichol J. L., Sullivan J. T. (2004). Pharmacodynamics and pharmacokinetics of AMG 531, a novel thrombopoietin receptor ligand. Clin Pharmacol Ther 76: 628-38.

Each reference cited in this specification is incorporated by reference in its entirety. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the claims that follow.

What is claimed is:

1. A method of treating idiopathic thrombocytopenia purpura (ITP) in a patient having ITP, which comprises:
a. Administering romiplostim weekly to the patient;
b. Increasing the weekly dose until a platelet count of at least about 50 to 200×10⁹/L is reached;
c. Decreasing the weekly dose of romiplostim if the platelet count remains ≥200×10⁹/L for two consecutive weeks;
d. Discontinuing romiplostim if (i) the platelet count has remained ≥200×10⁹/L for two consecutive weeks when the weekly dose is 1 μg/kg or (ii) the platelet count is ≥400×10⁹/L; and
e. If a platelet count ≥200×10⁹/L is reached within the first 4 to 12 weeks of treatment, maintaining a treatment-free period of at least about 24 weeks during which the patient receives no romiplostim.

2. The method of claim 1, which comprises administering an initial dose of 1 μg/kg of romiplostim to the patient.

3. The method of claim 1, which comprises maintaining the weekly dose so long as the platelet count is within about 50 to 200×10⁹/L.

4. The method of claim 1, wherein the platelet count is ≥200×10⁹/L in the first four weeks of treatment.

5. The method of claim 1, wherein the platelet count is ≥200×10⁹/L in the first eight weeks of treatment.

6. The method of claim 1, wherein the increase in the dose of romiplostim is in increments of 1 μg/kg weekly.

7. The method of claim 1, wherein the dose in step c is decreased in increments of 1 μg/kg.

8. The method of claim 1, wherein the patient's platelet count remains ≥50×10⁹/L during the treatment-free period.

9. The method of claim 1, wherein the patient receives no ITP medications during the treatment-free period.
